(19) United States
(12) Patent Application Publication
(10) Pub. No.: US 2016/0158066 A1
(43) Pub. Date: Jun. 9, 2016

(54) NEGATIVE PRESSURE WOUND DRESSING

(71) Applicant: BENQ MATERIALS CORPORATION, Taoyuan City (TW)

(72) Inventor: Shih-Wei CHAO, Taoyuan City (TW)

(21) Appl. No.: 14/789,554

(22) Filed: Jul. 1, 2015

(30) Foreign Application Priority Data
Dec. 9, 2014 (TW) 103221852

(51) Int. Cl. A61F 13/00; A61M 1/00; A61F 13/02

(52) U.S. Cl. CPC A61F 13/00063; A61F 13/00029; A61F 13/022; A61F 13/00068; A61M 1/0088; A61F 2013/00174

(57) ABSTRACT

A negative pressure wound dressing is provided. The negative pressure wound dressing sequentially includes a backing layer, a water blocking layer, an absorbent layer and a wound contacting layer. Wherein, the backing layer includes a connecting aperture for fluidly communicating to a negative pressure source. The wound contacting layer includes a surface layer for wound contacting and a plurality of chambers containing a medication, wherein openings of the chambers are disposed on the surface layer for releasing the medication contained in the chamber. The medication can be prevented from being suctioned to the absorbent layer and can be released toward the wound to enhance the wound healing by using the negative pressure wound dressing.

### RELATED APPLICATIONS

This application claims priority to Taiwanese Application Serial Number 103221852, filed Dec. 9, 2014, which is herein incorporated by reference.

### BACKGROUND

1. Field of Invention

The invention relates to a negative pressure wound dressing, and more particularly to a negative pressure wound dressing including a medication.

2. Description of Related Art

Wound healing and repair is a complex process where the skin or other body tissue heals and repairs itself after injury. Firstly, the body delivers proteins and other clotting substances to the wound by blood to minimize the injury. Blood clots are provided to stop bleeding, at the same time, white blood cells engulf bacteria and other pathogens or debris. And then, a healing phase called proliferation, the body begins to heal and repair itself, granulation tissue is formed on the epidermis. The granulation tissue on the wound provides a basic structure, and cells can grow from around to internal the wound to heal the wound. Finally, scars will form with time.

Conventional wound dressing absorbs blood and tissue fluid from the wound to maintain dryness of the wound by physical adsorption method. However, conventional wound dressing cannot control the environment humidity around the wound, and excessive dry environment will affect the wound healing.

A method for controlling the environment humidity to enhance wound healing in proliferation phase called negative pressure wound therapy (NPWT). Negative pressure wound therapy is a therapeutic technique using a simple negative pressure source, such as vacuum pump, combined with sealed drape, soft suction head and biocompatible porous material to form a negative pressure environment for extracting pus and infectious materials and increasing blood flow around the area to enhance the wound healing. The negative pressure wound therapy uses nature healing process of body to increase blood flow to the wound effectively, enhance granulation tissue forming on the wound and growing healthy tissue. The negative pressure wound therapy can inhibit the growth of bacteria by removing exudates fluid from the wound.

To enhance wound healing, repair factor, anti-bacterial or anti-inflammatory ingredients can be added into the conventional negative pressure wound dressing selectively. The above mentioned pharmaceutical ingredients can be coated on or formed as particles into the dressing fibers or wound contacting layer. However, no matter in the form of coating or particles, the coating or particle is easily shedded from the dressing, which will affect the gas flow and cause negative pressure provided by negative pressure source being non-uniform on the wound dressing. Besides, the shedding coating or particles might be suctioned with the tissue fluid into the absorbent layer or liquid-collection canister to obstruct the negative pressure delivery such that an expected repair effect cannot be reached.

In view of the foregoing, there is still a need for a negative pressure wound dressing containing a medication, which can be used in negative pressure therapy system, wherein the medication will be released toward the wound during continuously or intermittently negative pressure delivered by the system and not easily be suctioned by the negative pressure to obstruct the negative pressure delivery and to detriment the expected healing and repair effect.

### SUMMARY

The present invention is directed to a novel negative pressure wound dressing, wherein the medication can be prevented from being suctioned to the absorbent layer and can be released toward the wound to enhance wound healing by containing the medication in the chambers of the wound contacting layer.

According to an aspect of the present invention, a negative pressure wound dressing is provided. In an embodiment of the present invention, the negative pressure wound dressing includes a backing layer comprising a connecting aperture for fluidly communicating to a negative pressure source; a water blocking layer is disposed on the backing layer; an absorbent layer is disposed on the water blocking layer; and a wound contacting layer is disposed on the absorbent layer and comprises a surface layer for wound contacting and a plurality of chambers containing a medication, wherein openings of the chambers are disposed on the surface layer for releasing the medication contained in the chamber.

In an embodiment of the present invention, the diameter of the openings of the chambers is in a range of 0.1 μm to 100 μm.

In an embodiment of the present invention, the medication is selected from the group consisting of epidermal growth factor, platelet rich plasma, fibroblast growth factor, macrophage derived growth factor, fibrin, anti-oxidants, anti-microbial agents, vitamins, micro nourishing, antibiotics, antifungal agents and a combination thereof.

In an embodiment of the present invention, the wound contacting layer is a manifold layer.

In an embodiment of the present invention, the material of the wound contacting layer is selected from the group consisting of polyurethane, polyolefin, vinyl acetate, silicone, fluorosilicone, fluoroelastomers, styrene, butadiene and thermoplastic elastomer.

In an embodiment of the present invention, the absorbent layer is able to hold at least 30 milliliters of fluid.

In an embodiment of the present invention, the material of the absorbent layer is selected from the group consisting of fiber, sponge, foam material, super absorbent polymer, hydrogel material and gel material.

In an embodiment of the present invention, the material of the water blocking layer is selected from the group consisting of polyvinyl alcohol, polyvinyl acetate, polyvinyl pyrrolidone, polyurethanes, polyamides, polyesters, polyacrylates, polymethyl methacrylate and poly acrylamide.

In an embodiment of the present invention, the backing layer is an impermeable elastic film.

In an embodiment of the present invention, the material of the backing layer is selected from the group consisting of natural rubbers, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, polyurethane, ethylene vinyl acetate, co-polyester and silicones.

The forgoing presents a simplified summary of the disclosure in order to provide a basic understanding of the present invention. This summary is not an extensive overview of the disclosure and it does not identify key/critical elements of the present invention or delineate the scope of the present invention. Its sole purpose is to present concepts disclosed herein in a simplified form as a prelude to the more detailed description that is presented later.

Many of the attendant features will be more readily appreciated as the same becomes better understood by reference to the following detailed description considered in connection with the accompanying drawings. It is noteworthy that the drawings shown in the figures are for illustrative purposes only and not to scale.

### BRIEF DESCRIPTION OF THE DRAWINGS

The invention can be more fully understood by reading the following detailed description of the embodiment, with reference made to the accompanying drawings as follows:

FIG. 1 is a cross-sectional view of a negative pressure wound dressing according to an embodiment of the present invention.

### DETAILED DESCRIPTION

Accordingly, a negative pressure wound dressing is provided. In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

According to an aspect of the present invention, a negative pressure wound dressing is provided.

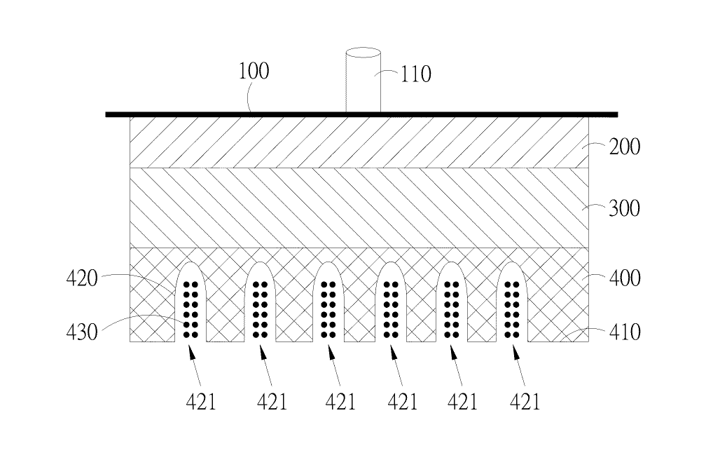

FIG. 1 is a cross-sectional view of a negative pressure wound dressing according to an embodiment of the present invention. The negative pressure wound dressing sequentially comprises a backing layer 100, a water blocking layer 200, an absorbent layer 300 and a wound contacting layer 400.

As used herein, “negative pressure” refers to a pressure less than the ambient pressure at a tissue site that is being subjected to treatment. In most cases, this negative pressure will be less than the atmospheric pressure at which the patient is located. Alternatively, the negative pressure may be less than a hydrostatic pressure at the tissue site. The negative pressure may be constant or varied and which may be delivered continuously or intermittently.

The backing layer 100 is disposed above a wound. In an embodiment of the present invention, the edge of the backing layer 100 near the wound side comprises an adhesive used to form a fluid seal environment between the backing layer 100 and the around epidermis of the wound. In another embodiment of the present invention, the backing layer 100 is adhered around the epidermis of the wound to form a fluid seal environment by an outside tape. Therefore, the backing layer 100 can be a microbial barrier to prevent pollutants from entering the wound and fix the absorbent layer 300 and the wound contacting layer 400 above the wound. The backing layer 100 comprises a connecting aperture 110 for fluidly communicating to a negative pressure source, such as a vacuum pump or another suction source.

Suitable material of the backing layer 100 can be a moisture permeable membrane to promote the exchange of oxygen and moisture between the atmosphere and the wound. The backing layer 100 can also be an impermeable elastic film or rigid member. To consider the convenience of use and the fits for wound, in an embodiment of the present invention, the backing layer 100 is an impermeable elastic film. In an embodiment of the present invention, the material of the backing layer 100 can be, such as rubbers, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, polyurethane, ethylene vinyl acetate, co-polyester or silicones.

The water blocking layer 200 is disposed on the backing layer 100. The water blocking layer 200 can prevent liquid from delivering but allow a gas (for example water vapor) to leave. The water blocking layer 200 can maintain the negative pressure environment in the dressing. Suitable material of the water blocking layer 200 is a material which can prevent fluid from entering and leaving through the material and allow gas to leave or flow through the material. The water blocking layer 200 can be but not limited to high water vapor transfer membrane or other structure formed from hydrophilic polymer. In an embodiment of the present invention, the material of the water blocking layer 200 can be polyvinyl alcohol, polyvinyl acetate, polyvinyl pyrrolidone, polyurethanes, polyamides, polyesters, polyacrylates, polymethyl methacrylate or poly acrylamide.

The absorbent layer 300 is disposed on the water blocking layer 200. The conventional negative pressure wound dressing must comprises a liquid-collection canister for containing a fluid which is suctioned by the negative pressure, such as tissue fluid. However, disposing a liquid-collection canister needs more space and may make the patients not easy to move. The liquid-collection canister can be replaced by disposing the absorbent layer 300, and the tissue fluid can be contained effectively. Moreover, the tissue fluid can stay away from the skin of patient to avoid impregnating the wound by containing the tissue fluid in the absorbent layer 300. In surgical application, when using the negative pressure wound therapy without disposing a liquid-collection canister, the absorbent layer 300 must has to be able to hold at least 30 milliliters of fluid. In an embodiment of the present invention, the material of the absorbent layer 300 can be but not limited to fiber, sponge, foam material, super absorbent polymer, hydrogel material or gel material.

The wound contacting layer 400 is disposed on the absorbent layer 300. The wound contacting layer 400 comprises a surface layer 410 for directly contacting with the wound. In an embodiment of the present invention, the wound contacting layer 410 is a manifold layer. The term “manifold” as used herein refers to a substance or structure that is provided to assist in applying negative pressure to, delivering fluids to, or removing fluids from a tissue site. A manifold typically includes a plurality of flow channels or pathways that are interconnected to improve distribution of fluids provided to and removed from the area of tissue around the manifold. The manifold is placed at the tissue site, which is a biocompatible material that is capable of being placed in contact with the tissue site and distributing negative pressure to the tissue site. Example of material used as manifold includes but not limited to materials that have structural elements arranged to form flow channels, such as cellular foam open-cell foam, porous tissue collections, liquids, gels and foams that include or cured to include flow channels. In an embodiment of the present invention, the wound contacting layer 400 is a porous foam and includes a plurality of interconnected cells or pores that act as flow channels. The porous foam can be polyurethane, open-cell, reticulated foam or open flexible polymeric foam, such as polyolefins, ethylene vinyl acetate, silicone, fluorosilicone, fluoroelastomers, styrene, butadiene or thermoplastic elastomer.

The wound contacting layer 400 comprises a plurality of chambers 420 and openings 421 of the chambers 420 are disposed on the surface layer 410 of the wound contacting layer 400, wherein the chambers 420 contain a medication 430. During operating under continuously or intermittently negative pressure, the medication 430 is contained in the chambers 420 and is protected by the chambers 420, thus the medication 420 will release toward the wound for healing the wound and not easily be suctioned far from the wound by negative pressure. In the embodiment, the diameter of the openings 421 of the chamber 420 can be adjusted by the releasing rate of the needed medication 430. In an embodiment of the present invention, the diameter of the openings 421 of the chambers 420 is in a range of 0.1 μm to 100 μm. The distribution density of the chambers 420 in the wound contacting layer 400 can be adjusted according to necessity and not affect the suction by negative pressure.

In order to control the infection of the wound effectively, debridement and promote wound healing, the medication 430 can comprises but not limited to epidermal growth factor, platelet rich plasma, fibroblast growth factor, macrophage derived growth factor, fibrin, anti-oxidants, anti-microbial agents, vitamins, micro nourishing, antibiotics or antifungal agents according to different necessity.

Accordingly, when using the negative pressure wound dressing of the present invention, the wound is fluid sealed by the backing layer 100, and the connecting aperture 110 of the backing layer 100 is fluidly communicated to a negative pressure source. The negative pressure is delivered uniformly to the wound through the wound contacting layer 400. The tissue fluid of the wound is suctioned into the absorbent layer 300, and the tissue fluid is blocked by the water blocking layer 200 and is contained in the absorbent layer 300. The medication 430 is protected by the chamber 420, thus, the medication 430 will release toward the wound and not easily be suctioned by the negative pressure. Therefore, the wound healing effect of the medication 430 can be improved. The negative pressure wound dressing of the present invention can increase the medication 430 residence time in the wound and promote the wound healing thereby.

The advantages of the negative pressure wound dressing of the present invention are as the following:

1. Compared to conventional negative pressure wound therapy, the negative pressure wound dressing of the present invention is more convenient to use and not affects the movement of patients because of without a need for additionally disposing a liquid-collection canister.

2. In addition to enhance wound healing by negative pressure wound therapy, the additional medication can prevent wound infection and improve debridement and wound healing effect.

3. The additional medication can surmount the disadvantages of medication shedding in conventional dressing and maintain the healing effect to the wound from the medication.

In the manner described above, the negative pressure wound dressing of the present invention can promote the medication to stay in the wound, and the medication will not be suctioned into the absorbent layer or liquid-collection canister easily to enhance the wound healing effectively.

While the invention has been described by way of example(s) and in terms of the preferred embodiment(s), it is to be understood that the invention is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A negative pressure wound dressing, comprising:
a backing layer comprising a connecting aperture for fluidly communicating to a negative pressure source;
a water blocking layer disposed on the backing layer;
an absorbent layer disposed on the water blocking layer; and
a wound contacting layer disposed on the absorbent layer, comprising a surface layer for wound contacting and a plurality of chambers containing a medication, wherein openings of the chambers are disposed on the surface layer for releasing the medication contained in the chamber.

2. The negative pressure wound dressing according to claim 1, wherein the diameter of the openings of the chambers is in a range of 0.1 μm to 100 μm.

3. The negative pressure wound dressing according to claim 1, wherein the medication is selected from the group consisting of epidermal growth factor, platelet rich plasma, fibroblast growth factor, macrophage derived growth factor, fibrin, anti-oxidants, anti-microbial agents, vitamins, micro nourishing, antibiotics, antifungal agents and a combination thereof.

4. The negative pressure wound dressing according to claim 1, wherein the wound contacting layer is a manifold layer.

5. The negative pressure wound dressing according to claim 1, wherein a material of the wound contacting layer is selected from a group consisting of polyurethane, polyolefin, vinyl acetate, silicone, fluorosilicone, fluoroelastomers, styrene, butadiene and thermoplastic elastomer.

6. The negative pressure wound dressing according to claim 1, wherein the absorbent layer is able to hold at least 30 milliliters of fluid.

7. The negative pressure wound dressing according to claim 1, wherein a material of the absorbent layer is selected from the group consisting of fiber, sponge, foam material, super absorbent polymer, hydrogel material and gel material.

8. The negative pressure wound dressing according to claim 1, wherein a material of the water blocking layer is selected from the group consisting of polyvinyl alcohol, polyvinyl acetate, polyvinyl pyrrolidone, polyurethanes, polyamides, polyesters, polyacrylates, polymethyl methacrylate and poly acrylamide.

9. The negative pressure wound dressing according to claim 1, wherein the backing layer is an impermeable elastic film.

10. The negative pressure wound dressing according to claim 1, wherein a material of the backing layer is selected from the group consisting of natural rubbers, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, polyurethane, ethylene vinyl acetate, co-polyester, and silicones.
